(19) United States
(12) Patent Application Publication
(10) Pub. No.: US 2023/0225614 A1
(43) Pub. Date: Jul. 20, 2023

(54) Wireless Veterinary Patient Monitor

(71) Applicant: VETRISENSE LTD., Tel Aviv (IL)

(72) Inventor: Avi YOSEF, Holon (IL)

(21) Appl. No.: 18/093,430

(22) Filed: Jan. 5, 2023

Related U.S. Application Data
(60) Provisional application No. 63/300,068, filed on Jan. 17, 2022.

(51) Int. Cl. A61B 5/00; A61B 5/08; A61B 5/256; A61B 5/27; A61B 5/28; A61B 5/273; A61B 5/332

(52) U.S. Cl. CPC A61B 5/002; A61B 5/0806; A61B 5/256; A61B 5/27; A61B 5/28; A61B 5/273; A61B 5/332; A61B 5/6823; A61B 5/0006; A61B 5/6831; A61B 2562/14; A61B 2562/227; A61B 2503/40; A61B 2562/164

(57) ABSTRACT

A veterinary vital signs monitoring system, including: (A) two stretchable bands, each having an electrically conductive component; (B) a housing including two fasteners with electrically conductive contacts, each fastener adapted to receive, and lock therein a portion of one of the bands, the conductive contacts adapted to make an electrical connection with electrically conductive components of the bands; and (C) a module housed in the housing and in electrical communication with the conductive contacts, the monitoring module adapted to receive signals relating to vital signs of the animal on which the bands are mounted, via the conductive components thereof.

### FIELD OF THE INVENTION

The present invention relates to a vital-signs monitor for animals and, more particularly, to a wireless veterinary patient vital-signs monitor.

### BACKGROUND OF THE INVENTION

Conventional patient monitors in human hospitals are heavy and bulky and use sensors that connect to a patient's body via wires. The patient's vital signs are displayed on screens by the patient's hospital bed and are also transmitted separately to displays by the nursing station. If a patient starts to fail physically the monitor will trigger visual and audible alarms to notify the nursing staff. This level of patient monitoring of humans has become the standard of care in nearly all hospitals worldwide.

While this form of patient monitoring might work with humans lying obediently in hospital beds it cannot work with veterinary patients who periodically stand up and/or rotate in their cages while hospitalized. When movement occurs, the sensors attached to the pets dislodge and false alarms are inevitably set off. Consequently, in veterinary hospitals patient monitors are used only when the companion pets (mostly dogs and cats) are sedated, immobile and/or in surgery under general anesthetic. Vital signs of non-sedated patients who are post-surgery or in critical condition are taken manually by the veterinary nurses as often as every 15 minutes after surgery.

### SUMMARY OF THE INVENTION

According to the present invention there is provided a veterinary vital signs monitoring system, including: (A) two stretchable bands, each having an electrically conductive component; (B) a housing including two fasteners with electrically conductive contacts, each fastener adapted to receive, and lock therein a portion of one of the bands, the conductive contacts adapted to make an electrical connection with electrically conductive components of the bands; and (C) a module housed in the housing and in electrical communication with the conductive contacts, the monitoring module adapted to receive signals relating to vital signs of the animal on which the bands are mounted, via the conductive components thereof.

According to further features the signals received via the conductive component are electrical signals produced by a heart of the animal. According to still further features a conductive gel or paste is adapted to be applied between a portion of each band and any two separate locations on the animal to enhance conductivity of the electrical signals between the heart and the conductive component of the band. According to still further features a portion of the band is secured in conductive contact with the skin of the animal. For example, end of the band may be made from conductive polymer and attached to skin using water and pressure. Alternatively, another fixation element such as molded polymer (conductive or not) may be positioned at the end of the band. According to still further features the electrical signals are adapted to be processed and/or rendered into an electrocardiogram (ECG) by the monitoring module. In embodiments, the ECG is displayed on a display on the housing or transmitted wirelessly to a remote display.

According to still further features the system further includes at least one vital signs sensor in wired or wireless communication with the module or embedded therein, the at least one sensor selected from the group including: an oxygen saturation (SPO2) sensor, a respiration sensor, blood pressure sensor, and a temperature sensor. According to still further features the system further includes at least one non-vital sensor in wired or wireless communication with the module or embedded therein.

According to still further features the module is adapted to fit between shoulder blades of the front legs of a four-legged animal. According to still further features the housing is adapted to be held in place by the stretchable bands placed around or near the chest area or forelegs of an animal.

According to still further features the system further includes a Respiratory Inductance Plethysmography (RIP) sensor including: respiration sensing belt comprised of a non-conductive stretchable material and conductive material disposed in or on the stretchable material, and a coupling piece adapted to mechanically and electrically couple together two ends of the respiration sensing belt once positioned around a torso of the animal; and an electronic interface adapted to obtain digital waveforms from the respiration sensing belt, via the coupling piece; wherein the RIP sensor is in wired or wireless communication with the monitoring module or coupled to the housing.

According to still further features the coupling piece includes two electrical contacts and wherein fastening ends of the respiration band to the coupling piece brings the conductive material into secure and unmoving contact with the electrical contacts. According to still further features the module housing further includes at least one additional conductive component.

According to still further features each of the stretchable bands is pre-cut or cut from a larger roll of a same material.

According to another embodiment there is provided a method of monitoring an animal, the method including: providing two stretchable bands, each band adapted to be coupled to the animal on one end thereof; providing a housing with fasteners for the stretchable bands; installing a module in housing; positioning the module between shoulder blades of forelegs of the animal, securing the bands to the animal, such that at least a portion of each band is in contact with a separate respective area of skin of the animal; fastening loose ends of the bands to the fasteners of the housing; providing at least one sensor on the animal, the at least one sensor being in electrical communication with the module; receiving sensor data at the module from the at least one sensor.

According to further features the method further includes wirelessly transmitting the received sensor data to a remote monitoring station.

According to still further features each material band has an electrically conductive material; wherein the fasteners include electrical contacts in electrical communication with the module; and wherein fastening the two material bands to the fasteners mechanically couples the bands to the housing and electrically couples the conductive material to the module.

According to still further features at least one sensor is selected from the group including: a temperature sensor, an oxygen saturation sensor, a blood pressure sensor, and a Respiratory Inductance Plethysmography (RIP) sensor.

According to still further features the RIP sensor includes: a respiration sensing belt comprised of a non-conductive stretchable material and conductive material disposed in or on the stretchable material, and a coupling piece adapted to mechanically and electrically couple together two ends of the respiration sensing belt once positioned around a torso of the animal; wherein the RIP sensor is in wired or wireless communication with the module.

According to still further features the method further includes applying a conductive gel or paste to the separate respective areas of skin of the animal prior to securing the bands to the animal such that the conductive components are in electrical contact with the areas of skin to enable electrical signals produced by the heart of the animal to be conducted to the module via the conductive components.

According to still further features at least one additional sensor is attached to the animal and in electrical communication with the module.

According to still further features at least one additional sensor is selected from the group including: a temperature sensor, an oxygen saturation sensor, a blood pressure sensor, and a Respiratory Inductance Plethysmography (RIP) sensor.

According to still further features the RIP sensor includes: a respiration sensing belt comprised of a non-conductive stretchable material and conductive material disposed in or on the stretchable material, and a coupling piece adapted to mechanically and electrically couple together two ends of the respiration sensing belt once positioned around a torso of the animal; wherein the RIP sensor is in wired or wireless communication with the monitoring module.

### BRIEF DESCRIPTION OF THE DRAWINGS

Various embodiments are herein described, by way of example only, with reference to the accompanying drawings, wherein:

FIG. 1 is an exemplary animal, a dog, wearing a wireless veterinary monitoring system 100 including two material elastic bands coupled to a housing;

FIG. 1A is a magnified and top-down view of a portion of the system 100;

FIG. 2A is an example diagram of a housing including fastening/clasping means and module;

FIG. 2B is a schematic diagram of an example conductive band;

FIG. 3A is an example housing in an open state without bands;

FIG. 3B is a view of the housing 300 in an open state in which the straps 120L and 120R are thread through the left- and right-hand slots 342, 346 respectively;

FIG. 3C is a view of housing 300 in a closed state in which the upper section is closed over the lower section and the bands are in electrical communication with the module;

FIG. 3D is a view of the housing in the closed state with the lengths of the bands visible;

FIGS. 4A-D are views of an example embodiment of a buckle with conductive contacts;

FIG. 5 is a possible configuration of the assembly;

FIG. 6 is another configuration of the assembly.

### DESCRIPTION OF THE PREFERRED EMBODIMENTS

The principles and operation of a veterinary vital-signs monitor according to the present invention may be better understood with reference to the drawings and the accompanying description.

The word “buckle” as used herein is intended to convey the meaning of a fastener that fastens together two ends of a belt or strap.

The word “clasp” as used herein is intended to convey the meaning of “a device for fastening together two or more things or parts of the same thing”.

In the instant disclosure, there is disclosed a buckle with at least two clasps. Each clasp secures one end of a material band. The buckle forms part of a housing which houses a module (also referred to herein as a “monitoring module”, “data logger” and variations thereof).

The instant system uses a band that is made of material that is elastic or stretchable. The terms “band”, “strap”, “belt”, “elastic band [of fabric material]”, “stretchable retention bands”, “elastic straps”, and similar terms are used interchangeably herein to refer to flexible/stretchy material that is able to resume its normal shape spontaneously after dilatation, contraction, or distortion.

A general term “harness”, and variations thereof, are used herein to refer the band(s) and the fasteners (e.g., buckle(s) and clasps) as well as the housing, to the exclusion of the monitoring module and additional sensors.

In some embodiments, the bands of the instant system include a conductive thread woven in the stretchable bands.

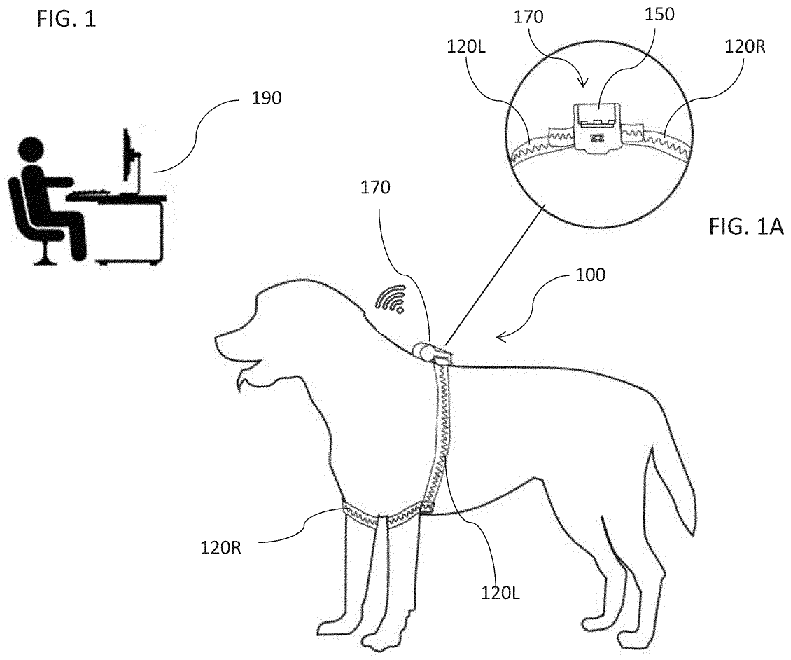

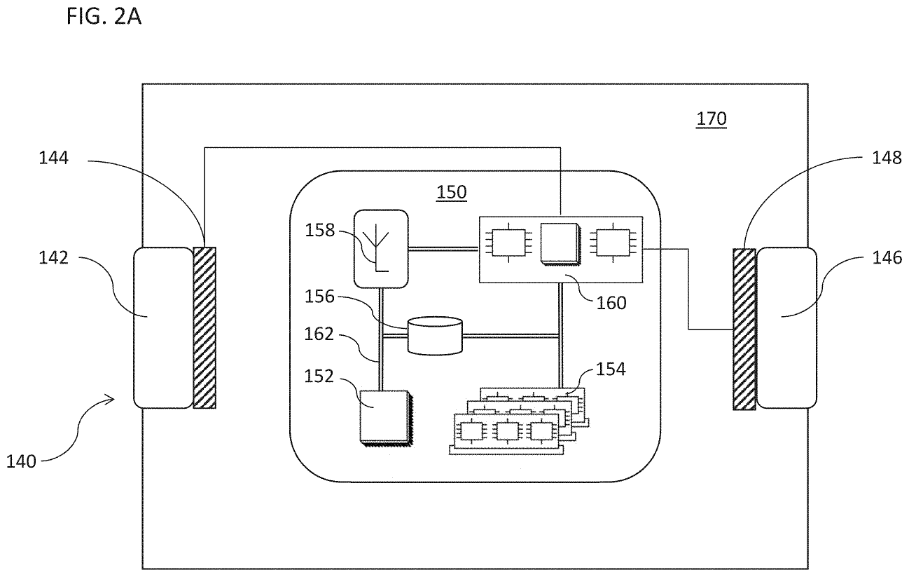

Referring now to the drawings, FIG. 1 illustrates an exemplary animal, a dog, wearing a veterinary monitoring system 100 including two material stretchable/elastic bands 120R and 120L, coupled to a housing 170. In the example embodiment depicted in the Figure, the system is in wireless communication with a monitoring station 190. In other embodiments (not shown) the system is in wired communication with the remote monitoring station. In the example embodiment depicted in the Figure, the housing is fastened between the shoulder blades of the animal. In the example embodiment, the stretchable bands hold the housing in place. In other embodiments, the housing may be held in any convenient place by any means known in the art. In the example embodiment, each band is wrapped around one of the forelegs, running under the armpits of the animal. In other embodiments, the bands may be adhered to the animal's body in other ways. The stretchable/elastic bands of material include a conductive component which is embedded in, or adhered onto, or otherwise part of the material. The conductive components of the bands must be in contact with the skin of the animal in two separate locations on the body of the animal. For example, on the chest, leg, armpit, etc.

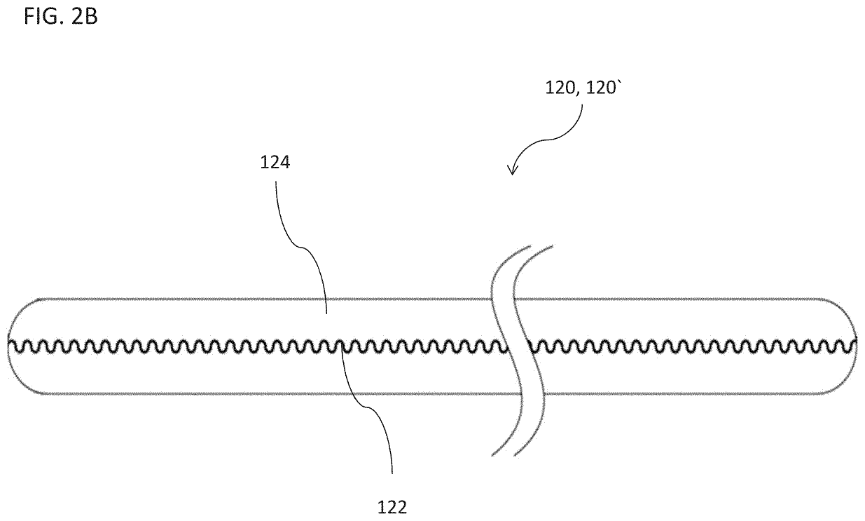

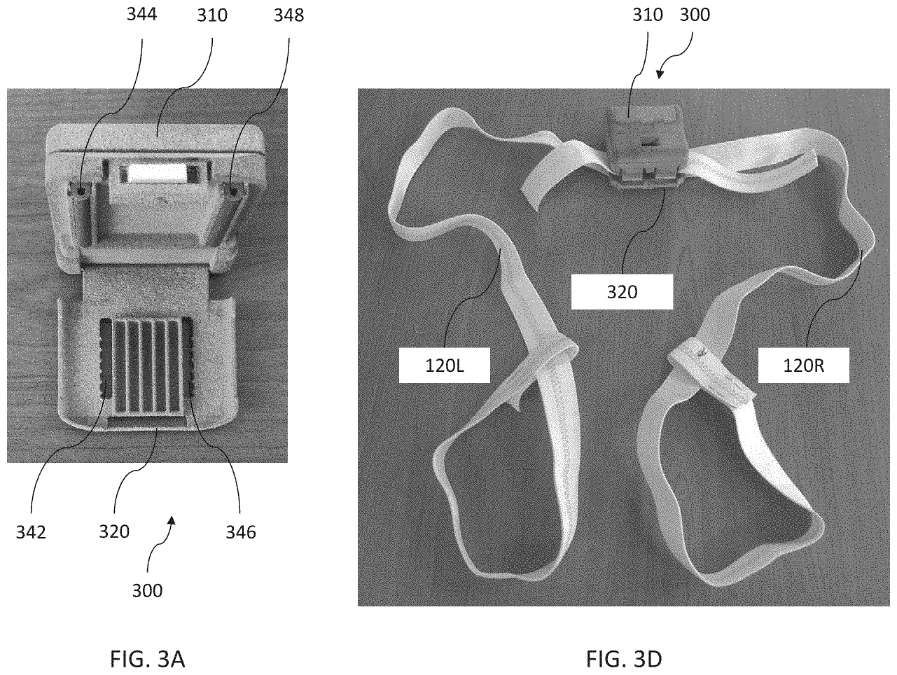

FIG. 1A is a magnified and top-down view of a portion of the system 100. A module/data logger/monitoring module 150 (not visible in FIG. 1, but schematically depicted in FIG. 2), is housed inside the housing 170. The housing is fitted with clasping and/or fastening means that mechanically fasten the material bands to the housing and electrically conductive contacts that are adapted to electrically couple the conductive components to the module.

Each of these components will be described in further depth below. In other embodiments of the system, additional components are added to the system, with all signals/sensor data being fed to the ‘brain’ of the system, the monitoring module. In embodiments, the module processes the signals received. In some embodiments, the module merely transmits the signals to a remote monitoring computer. In some embodiments the module processes some of the signals prior to transmitting them, and transmits some of the signal without processing them.

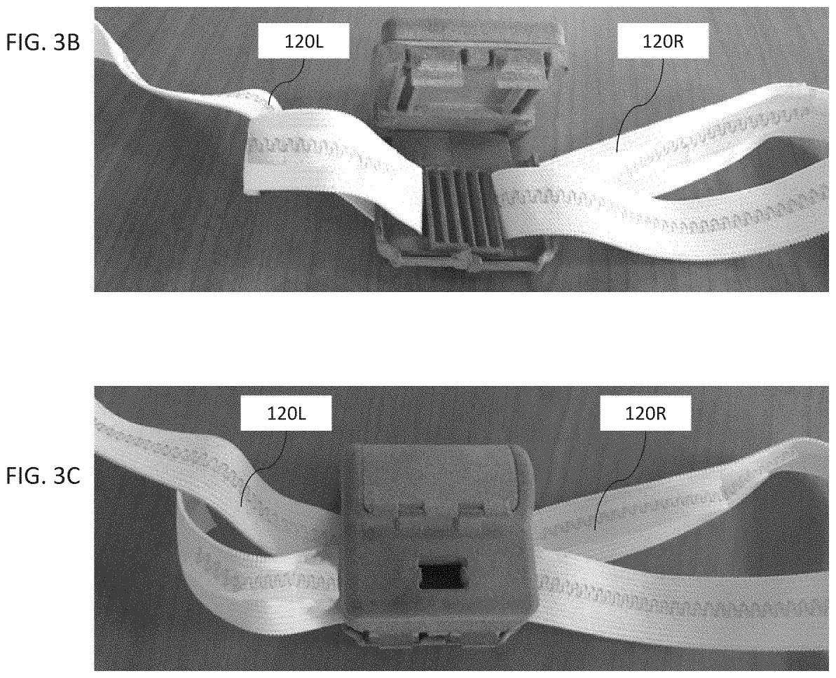

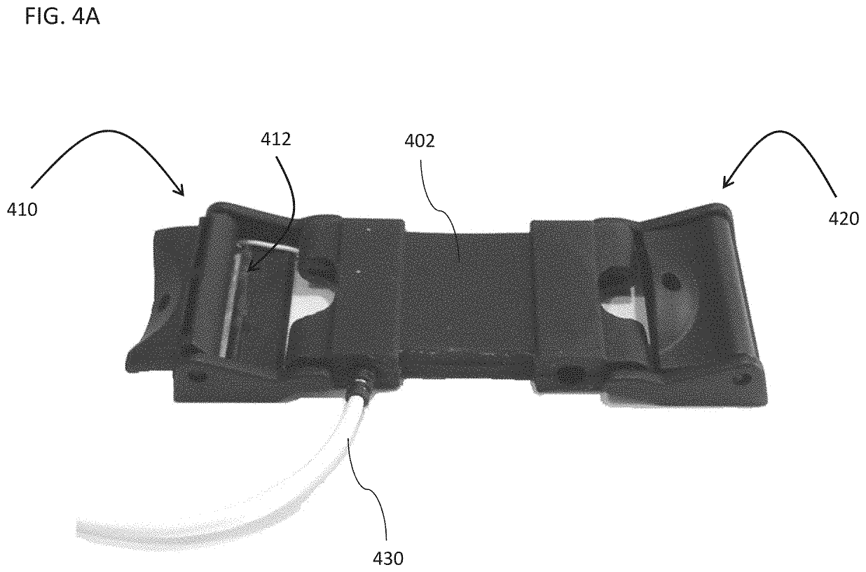

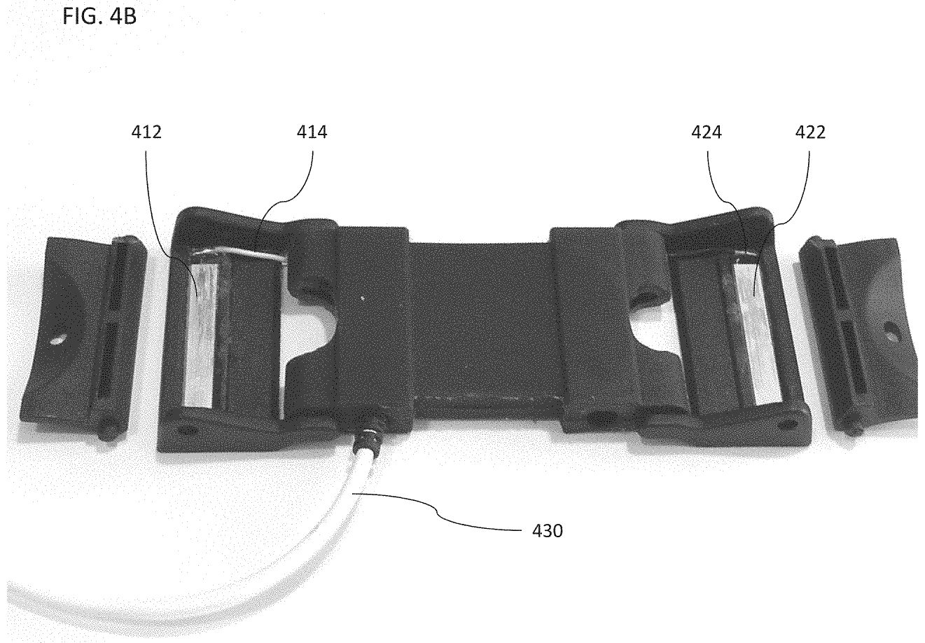

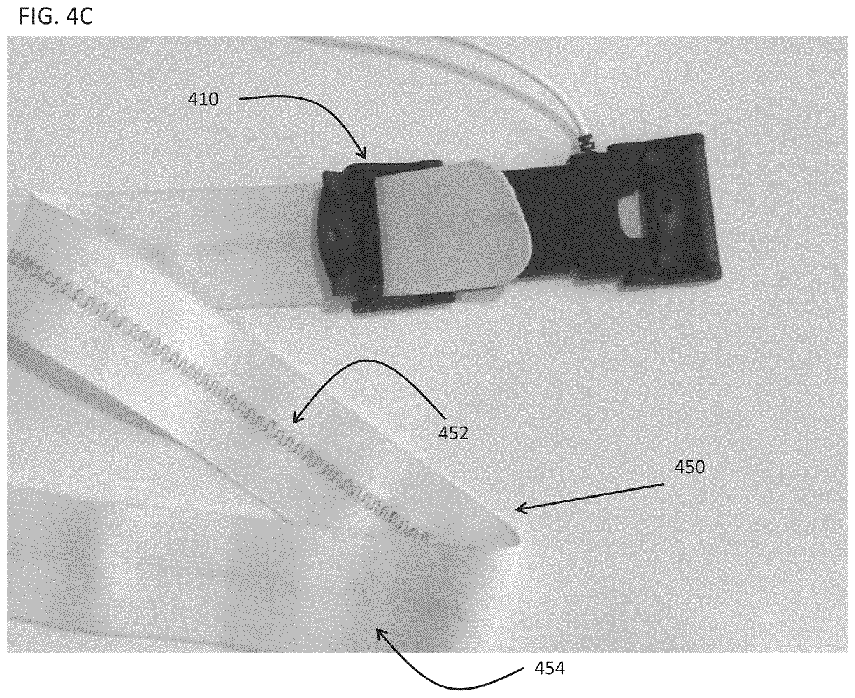

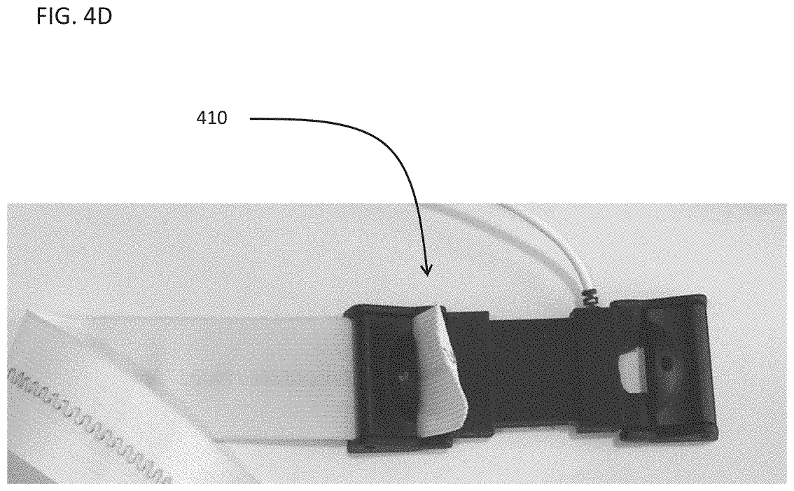

FIG. 2A illustrates an example diagram of a housing 170 including fastening/clasping means 140 (hereafter also referred to as “fastener” or “clasp”) and monitoring module 150. The term “fastening means” (and its alternatives and derivatives) include any type of arrangement that, on the one hand, mechanically holds the bands in place, and, on the other hand, makes an electrical connection with the conductive component of the band. One example embodiment is schematically depicted in FIG. 2A. Fastener 140 includes two clasps 142 and 146 on the left and right sides respectively. The left clasp is collocated with a left contact 144 and the right clasp 146 is collocated with a right contact 148. The clasp holds the belt or band as well as bringing the conductive element of the band into electrical communication with the contact 144, 148. Numerous variations of clasp mechanisms can facilitate the dual functionality. One variation is discussed in further detail below with reference to FIGS. 3A-3D and a second variation is discussed in further detail with regards to FIGS. 4A-4D. However, it is made clear that any mechanism that holds the band and forms an electrical circuit with conductive element in the band is considered to be within the scope of the invention. In some embodiments, more than two fasteners and/or conductive components may be included in the housing. For example, a third band for DRL contact to get better ECG signal (see below).

In an example embodiment, the data logger/monitor 150 includes a processing unit, e.g., processor 152 and memory 154 as well as storage 156, a wireless communication module 158, an input/output interface 160, all of which are connected via a bus 162. See below for additional details. It is made clear that the components and configuration are merely examples and can be implemented in many other ways, as would be obvious to those skilled in the art.

### Wireless Communication

Data from the monitoring module 150, e.g., physiological data in example embodiments, is transmitted (continuously or at given intervals or when attention is needed) wirelessly from the monitoring module (interchangeably referred to herein as the “monitor”, “module”, “data logger”, or simply “logger”) 150 to the nursing station (or to any other designated computer device on and/or offsite) 190 by the wireless communication module 158 via any applicable wireless communications protocol/wireless network, but most commonly via WiFi or Bluetooth. In some embodiments, the data is always, or at least sometimes, locally recorded on the data logger. Alternatively, or additionally, the data is stored on a storage device which is part of, or in communication with, a wireless network (not shown). According to another embodiment, the data may be stored for the duration of the time that the animal is monitored, or part thereof, and then transferred to the system via a wired or wireless manner.

For example, when the monitor is within range of the clinic's WiFi signal, the data is communicated to the nursing station (or other device designated to receive the information). When functioning in this manner, the data may or may not be recorded/stored locally on the monitor. In embodiments, the monitoring data is stored remotely and/or locally. According to some embodiments, if the monitor leaves the covered communications area, for e.g., when the animal is taken for a walk outside the veterinary clinic, then the data continues to be logged on the monitor but is only communicated to the nursing station once the monitor comes back into range and reconnects to the wireless network in the clinic.

The data, in example embodiments, is displayed in a similar manner in which vital signs are displayed in human hospitals (graphs, numerical values, etc.). If a veterinary patient is in physiological distress the nursing staff (or other designated person or people) will be alerted, e.g., via an audible alarm and/or a visual alarm. The present technology elevates the state of veterinary patient monitoring and brings it in line with current monitoring standards in human hospitals.

The bands 120R, 120L and fasteners 140 serve in a multifunction capacity: (1) hold logger in place; and (2) electrically connects the conductive thread in the band to the monitor. The bands and fasteners will be discussed hereafter.

### Conductive Band

FIG. 2B illustrates a schematic diagram of an example conductive band 120L/R. A conductive textile is a fabric which can conduct electricity. Conductive textiles can be made with metal strands woven into the construction of the textile. Conductive fibers consist of a non-conductive or less conductive substrate, which is then either coated or embedded with electrically conductive elements, often carbon, nickel, copper, gold, silver, or titanium. Substrates typically include cotton, polyester, nylon, and stainless steel to high performance fibers such as aramids and PBO. In embodiments, the bands can be woven from electrically conductive yarn.

The immediate bands 120R, 120L each include at least one conductive thread 122 woven into, or stitched onto, one or both sides of the material band 124, in a sinusoidal wave pattern. The conductive material may alternatively be otherwise mechanically attached to the band in any manner that allows for the stretching of the band without compromising the conductivity of the material. One alternative example is conductive ink printed in a sinusoidal wave pattern on the material.

Alternatively, the conductive material can, itself, be elastic and then the material can be attached in a straight line which stretches together with the elastic band. Exemplarily, the conductive thread can be made of intertwined steel and nylon fibers. Other combinations of less conductive (or non-conductive) and conductive fibers spun into a single thread are known in the art. Any conductive material or combinations of materials need to meet the following requirements: (a) has sufficiently low resistance to electrical current to match the application; (b) is sufficiently flexible to withstand repeated pulling and bending and maintain conductivity; and (c) can withstand strong pulling forces without tearing.

Alternatively, the conductive material can be printed, coated or sprayed on to the elastic material. Any manner of attaching conductive material to the band/belt is considered to be included within the scope of the invention.

Alternatively, a non-insulated wire can be threaded inside the thickness of the belt, and the buckle (with conductive clasp) can be equipped with conductive, penetrating teeth that pierce the band and make contact with the wire—also at any point along the band.

According to one alternative, the conductive thread/material is stuck, woven and/or formed on one side of the band. According to another alternative, the conductive material is provided on both sides of the elastic band. According to the former embodiment, care must be taken to ensure that the conductive material is inserted into the buckle such that the electrical contacts inside the buckle touch the conductive material. In the latter embodiment, no matter which way the band is inserted into the buckle, the electrical connection will be made. Additionally, the conductive material on the one side can be electrically connected to the conductive material on the other side, in case the band is twisted.

The band can be coupled to a contact (e.g., closed in the conductive clasp of the buckle) at any point along the band. Accordingly, the band does not need to be an exact size (although it can be precut to preferred general sizes). In fact, the band can come in a large volume roll of material, and the person dispensing the band (e.g., in a veterinary hospital or clinic setting, an orderly or nurse) can measure off a desired length and cut the strip off the roll. Alternatively, a mechanical/electronic dispenser dispenses one of a number of predetermined lengths (e.g., small, medium and large), according to a selection (such as a pushbutton or lever etc.).

That is to say that any material and method of application of the material that allows a conductive buckle to be attached to any point on the belt, without prior preparation of that point is considered to be within the scope of the invention.

### ECG Functionality

A function of the instant technology is that the harness (bands, fasteners and housing) and monitoring module can be used as an ECG monitor. An electrocardiogram (ECG) is a simple test that can be used to check the heart's rhythm and electrical activity. Sensors attached to the skin are used to detect the electrical signals produced by your heart each time it beats. The instant bands 120R, 120L can be used to essentially function as ECG electrodes. The conductive threads 122 (in or on the band —see above and below) makes continuous contact with the veterinary patient's skin. The conductive component/thread in the band receives/senses/registers cardiac pulses in the same way as electrodes attached to the body do. The bands may be made to be in contact with the skin of the animal in any manner.

In some embodiments, e.g., to enhance signal pickup (or ensure, in the first place, that the signal is registered via the conductive thread), a conductive gel or paste is applied between the subject (animal) and the band. The use of conductive gel and/or paste increases signal conductivity (from the body to the band). In some cases, the conductive gel or paste also serves to hold the band in place as the gel/paste has a sticky characteristic to it. The electric pulse created by each contraction of the heart is transmitted to the monitor through the conductive threads. As such, the band functions as an ECG electrode, without the need for additional, distinct electrodes.

In embodiments, the system relies on the “patient's” insulating fur layer to prevent electrical contact between the conductive element in/on the band and the body at any point except the location where fur was shaved off, and/or a conductive gel was applied, and/or any other method was employed to ensure conduction. As mentioned, a portion of each belt must be in electrical contact with the skin of the animal in at least two separate locations. These locations can be the chest, armpit, leg, or any other relevant location.

In embodiments, the electrical signals are adapted to be processed and/or rendered into an electrocardiogram (ECG) by the module. In embodiments, the housing further includes a display for displaying the ECG. In other embodiments, the raw signals (or indications thereof) are transmitted to a monitoring computer and rendered into an ECG on the monitoring computer.

The harness (arrangement of bands), as described, holds the data logger/monitoring module in place to receive sensor data. In embodiments, the bands 120R, 120L with the conductive elements 122, are wrapped around the forelegs of the animal (near the armpit) and adapted to function as electrodes. In some embodiments, additional bands may be used. For example, a third band may be used as a DRL contact to get better ECG signal. A Driven Right Leg (DRL) circuit, also known as Right Leg Driving technique, is an electric circuit that is often added to biological signal amplifiers to reduce common-mode interference. In such cases, the housing includes at least one additional conductive component for receiving electrical impulses from the one or more additional bands and/or conductive components. If necessary, additional fasteners may also be included in the housing.

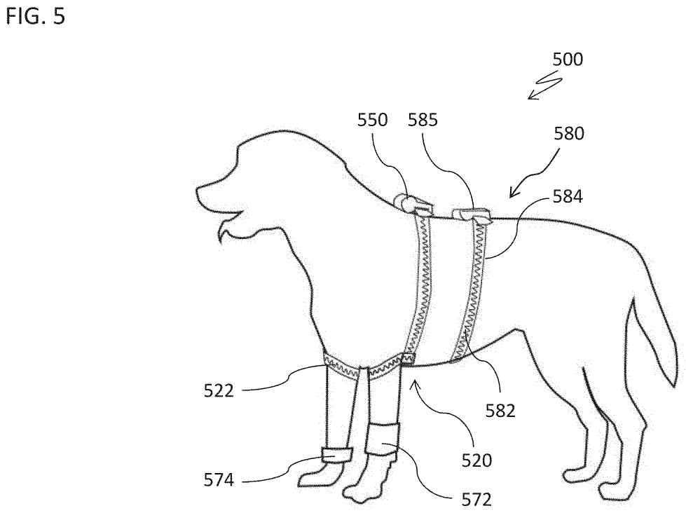

In embodiments, one or more addition vital signs and/or non-vital signs sensors may be in electrical communication with the module and/or embedded therein. The sensors may be in wired and/or wireless communication. Vital signs sensors include, but are not limited to heart rate monitor, blood pressure monitor (e.g., cuff 572—FIG. 5), SpO2 (Oxygen saturation) monitor (e.g., monitor 574—FIG. 5), respiration rate sensor, and a temperature sensor (e.g., core body temperature, and/or skin temperature).

Non-vital signs sensor sense physical and/or physiological parameters which are not necessarily “vital signs”. These non-vital signs sensors include but are not limited to: body motion/acceleration sensors, spatial orientation sensors, and other motion sensors, as well as ambient temperature or relative humidity (RH) sensors and the like.

### Buckle—Example I

One example embodiment of the buckle is depicted in FIGS. 3A-3D. FIG. 3A depicts an example housing in an open state without bands. According to the example embodiment, there is a housing 300 that has a clam-shell layout with an upper section 310 and a lower section 320 hinged together. The module 150 is seated in the upper section 310. The lower section 320 includes fasteners. In the example embodiment, there is a left-hand slot 342 and a right-hand slot 346, each for receiving one end of the bands 120R, 120L therethrough. Each slot includes teeth to increase the friction on the strap/band that is thread through the slot, in order to help hold the strap in place, prior to clamping the upper section closed over the lower section.

The upper section 310 includes electrical contacts 344 (left) and 348 (right) which are electrically coupled to the monitor module. In the depicted example embodiment, the contacts are conductive rubber tubes. When the upper section 310 is closed over the lower section 320, the bands are sandwiched between the contacts and the bottom surface of the lower section. The bands are held tightly by the contacts, which also form electrical engagement with the conductive component at the same time.

FIG. 3B depicts the housing 300 in an open state in which the straps 120L and 120R are thread through the left- and right-hand slots 342, 346 respectively. In the example embodiment, the bands have conductive threads on both sides of the bands. This type of band makes it easy for the veterinary staff to thread the bands into the buckles without having to worry which side the electric contact will come into contact with. Conversely, in a variation of the instant embodiment, a conductive strip or conductive rubber (or some other electrically conductive material) can be located above the band as well as under the band. That way, when the upper section is closed over the lower section contact can be formed with the conductive element of the elastic band irrespective of which way the band is facing. FIG. 3C depicts the housing 300 in a closed state in which the upper section is closed over the lower section and the bands are in electrical communication with the monitoring module.

FIG. 3D depicts the housing in the closed state with the lengths of the bands visible. In the example embodiment, each band 120L and 120R is arranged in with an adjustable loop at the distal end of the band. It is made clear that other manners of affixing the bands to the animal are considered to be within the scope of the invention. In use, the loop is adapted to be fit around a leg of an animal (veterinary patient) and pulled up tight under the armpits of the forelegs. In use, the loops are placed on the forelegs. Once fitted tightly around the forelegs, the loose ends of the straps are thread through the buckles of the open housing and then the housing is closed over a portion, e.g., the loose ends, of the bands and the assembly/system is ready for use.

### Buckle—Example II

An example embodiment of a buckle with conductive contacts is depicted in FIGS. 4A-4D. A variation of the buckle in the FIGS. 4A-4D is employed in the instant system, whereby the buckle is part of the housing that holds the monitoring module. As opposed to the configuration in FIGS. 4A-4D, the buckle in the instant system is in electrical communication with the monitoring module, feeding sensor data directly into the module.

Referring now to the second example buckle in FIGS. 4A-4D, FIG. 4A depicts a buckle 400, including a left clasp 410, a right clasp 420 and a connecting piece 402. One end of the band goes through the left clasp 410 and the other end through the right clasp 420. The band is pulled snug against the body (as needed) by pulling excess band through the eyelets and closing the clasps over the band. The clasps include one or more metallic contacts, coupled to the non-moving part of the buckle that pinches the strip/band in place.

In FIG. 4A, a metallic contact 412 is barely visible under the (open) moveable piece of the left clasp 410. The right clasp 420 is shown in the closed state. In other embodiments, the contact is not part of the pinching mechanism, but rather once the buckle is closed the conductive material comes into contact with the buckle contact. The buckle can also have more than one contact for coupling to both sides of the strip. In such an embodiment, even if the conductive material is only on one side of the band, it does not matter which side is inserted into the buckle.

In another embodiment, the buckle is completely metal. In another embodiment the metallic buckle is coated in plastic or some other non-conductive material. The parts that come into contact with the strip are left uncoated.

An electrical wire 430 is in electrical communication with the contacts 412 and 422. The electrical wire is adapted to conduct the electrical pulses received with the contacts to computing device. In the instant system, the contacts in the clasps may be in communication with monitoring module via the same or different electrical conduit(s).

FIG. 4B illustrates the buckle of FIG. 4A, with the clasps 410 and 420 disassembled. With the rotational component of the clasp removed from the setting, the left and right contacts 412 and 422 are clearly visible. Also in evidence are a left connecting wire (white) 414 and a right connecting wire (black) 424. The wires 414, 424 run through an insulated lead cord to the monitoring device. By coupling both ends of the belt/band to the coupling piece, the electric circuit is completed, and the monitoring device starts receiving signals.

FIG. 4C illustrates a band of material 450 with a conductive thread 452 inserted into the open clasp 410 of the buckle on the left side. The conductive thread 452 is facing the contact 412 inside the clasp. The side of the strip that includes the conductive thread is placed against the body. The blank side 454 of the band faces outwards. A medical technician placing the band on the animal tightens the band as desired by pulling excess material through the clasp slot, and then closing the clasp over the band.

FIG. 4D illustrates the left clasp in a closed position with the band caught in the clasp. Closing of the clasp 410 on the band 450 performs two functions: the first function is to physically restrain the band in the correct place (length); the second function is to create an electric contact between the band and the coupling piece. A protruding section of the clasp sandwiches the band inside the clasp, mechanically holding the band in place as well as electrically coupling the conductive thread 452 with the contact 412. The bands/belts are cheap to produce and very easily arranged on the patient. Typically, the bands will be used on a single patient and thrown away.

Another possible configuration of the assembly is shown in FIG. 5. According to example embodiments, there is disclosed an assembly/system 500 including a band 520 having a conductive element 522 (e.g., conductive thread) and non-conductive material 524. The band 520 is in mechanical and electrical communication with a monitoring module 550 (housed in a housing provided with one or more buckles/clasps, referenced collectively and/or interchangeably as monitoring module/housing/buckle(s)/clasp(s) 550), that is similar to the assembly/system 100. The detailed description provided for the embodiment of system 100 is to be seen as if repeated here in full, mutatis mutandis. In the instant configuration, the assembly/system 500 further includes one or more additional sensors. The sensor or sensors are placed on the animal and are in communication with the monitoring module or embedded therein.

The communication may be via a wireless connection or via a wired connection. In embodiments, signals may be communicated via the conductive element of the band to the monitoring module.

In the example depicted embodiment, the data logger 550 is held in place by the strap 520 in a position on the animal that is both secure and not easily dislodged by the animal. In embodiments, the logger transmits signals indicative of the vital signs data gathered by the data logger in a wired manner. In embodiments, the logger includes a wireless communications module for transmitting wireless signals indicative of the vital signs data gathered by the data logger. The data is transmitted wirelessly to a remote device for monitoring. The remote device may be collocated with the animal and/or remotely located. The signal may be transmitted via Wi-Fi and/or any other relevant wireless protocol. The wireless monitoring module may be a node/device on a network (not shown) and the data thereon may be viewed, retrieved, received, and/or manipulated by other devices on the network (e.g., a nurse's station or tablet computer) or in communication with the network (e.g., a veterinarian located remotely by able to log into the network from the remote location).

The data logger 550 is in electrical and mechanical communication with the band including a conductive element which provides ECG sensor readings as described above for system 100. In addition, the module 550 can be in communication with at least one additional sensor disposed on the animal. FIG. 5 depicts a plurality of such sensors. These sensors are merely exemplary both in their location and in their type. More or fewer or different sensors may be used. One or more sensors may be embedded in the housing itself.

That being said, common vital signs sensors used for monitoring veterinary patients include, but are not limited to: heart rate monitor, blood pressure monitor (cuff) 572, SpO2 (Oxygen saturation) monitor 574, respiration rate sensor, and a temperature sensor (e.g., core body temperature, skin temperature and/or ambient temperature). Additional sensors that are not vital signs sensors may also be connected to the module. Examples of physical or physiological parameters which are not “vital signs” include but are not limited to: body motion/acceleration, spatial orientation, and other motion sensors, as well as ambient temperature or relative humidity (RH) sensors.

Temperature sensors are preferably located on or in proximity to the underarm or underarms of one or both forelegs. In embodiments, a temperature sensor may be incorporated into, or attached to, band 520, at the preferred location.

The ECG band 520 generally needs to be adhered to the skin of the animal with a conductive (and preferably temporarily adhesive) material such as a gel. The band may be coated with an adhesive layer as well as conductive material. In some embodiments, the adhesive layer is also the conductive material. In some embodiments, the band and/or the other sensors may further include other or additional sensors, e.g., such as those listed above.

The sensors are in electrical communication (by wired or wireless means) with the monitoring module 550. In example embodiments, a wire or lead from the BP cuff, temperature (e.g., core body temperature, skin temperature and/or ambient temperature) and/or SPO2 sensor can be run along (on top of, under, inside, proximal to) the strap 520 to the monitoring module. In embodiments, the sensor or sensors are in wireless communication with the monitoring module 550. In embodiments, some sensors are in wired communication and other are in wireless communication.

In some embodiments, assembly/system 500 further includes a respiration rate sensor. One example is a Respiratory Inductance Plethysmography (RIP) sensor 580. The RIP sensor may be used in conjunction with the ECG band 520, and/or in conjunction with other sensors (e.g., SPO2, temperature, BP, etc.).

### Respiratory Inductance Plethysmography (RIP)

RIP is a method of evaluating pulmonary ventilation by measuring the movement of the chest and/or abdominal wall. Accurate measurement of pulmonary ventilation or breathing often requires the use of devices such as masks or mouthpieces coupled to the airway opening. These devices are often both encumbering and invasive, and thus ill-suited for continuous or ambulatory measurements, or for veterinary purposes. The instant RIP sensor 580 is an alternative RIP device that senses respiratory excursions at the body surface that can measure pulmonary ventilation.

The respiratory inductance plethysmography (RIP) sensor 580 includes a band 584, also referred to herein also as “respiratory band” or “transducer band” and an electrically conductive buckle 585. In an example embodiment, the respiration band 584 consists of a sinusoid wire 582 placed within a 2.5 cm (about 1 inch) wide, lightweight elastic band. In example embodiment, the wire may alternatively by a conductive thread woven into, or stitched onto, one side of the material band, in a sinusoidal wave pattern. In embodiments the conductive material may alternatively be otherwise mechanically attached to the band in any manner that allows for the stretching of the band without compromising the conductivity of the material. One alternative example is conductive ink printed in a sinusoidal wave pattern on the material.

In an alternative embodiment, conductive material can be mechanically coupled to both sides of the elastic band. In such an embodiment, no matter which way the band is inserted into the buckle, the electrical connection will be made. Additionally, the conductive material on the one side can be electrically connected to the conductive material on the other side, in case the band is twisted.

The transducer/respiratory band 584 is placed around the torso of the animal. The band is connected to an electronic interface adapted to obtain digital waveforms. In example embodiments, the electronic interface includes an oscillator and subsequent frequency demodulation electronics.

During inspiration the cross-sectional area of the rib cage and abdomen increases altering the self-inductance of the wire/conductive material and the frequency of their oscillation, with the increase in cross-sectional area proportional to lung volumes. The electronics convert this change in frequency to a digital respiration waveform where the amplitude of the waveform is proportional to the inspired breath volume.

The respiration band 584 of the immediate invention includes a conductive thread/material which is coupled onto one, external, side of the band such that the band can be coupled to an electrical contact at any point along the band. Therefore, the band does not need to be an exact size (although it can be precut to preferred general sizes). In fact, the band can come in a large volume roll of material, and the person dispensing the band (e.g., in a hospital setting, an orderly or nurse) can measure off a desired length and cut the strip of the roll. Alternatively, a mechanical/electronic dispenser dispenses one of a number of predetermined lengths (e.g., small, medium and large), according to a selection (such as a pushbutton or lever etc.).

The two ends of the band 584 are fastened together by a buckle 585. One such buckle has been detailed at length above with reference to FIGS. 4A-4D. That type of buckle, or variations thereof, can be used to fasten the ends of the respiratory band in both a mechanical and an electrical coupling. Coupling piece 585 includes two electrical contacts (not shown but similar to contacts 412 and 422 in FIG. 4), one contact for each end of the band. The ends of the band are secured by trapping the band in a fastener (e.g., a clasp similar to clasps 410 and 420) and thereby bringing the conductive material 582 into secure and unmoving contact with the electrical contacts of the coupling piece/buckle 585. By coupling both ends of the belt/band 584 to the coupling piece 585, the electric circuit is completed, and the monitoring module 550 starts receiving signals.

The band is connected to an oscillator and subsequent frequency demodulation electronics to obtain digital waveforms. These electronics may be housed inside the buckle 585 and transmitted to the monitoring module in a wired (not shown) or wireless manner. Alternatively, the oscillator and demodulation components may be housed in the monitoring module 550 and the respiration sensing belt directly connected to the module housing (not shown—but discussed below in further detail). In still another alternative embodiment, the data may be transmitted directly to the remote monitoring computer device (e.g., at the nurse's station/display/tablet computer, etc.) in parallel to the data transmitted from the data logger/monitoring module 550. In embodiments that include wireless transmission of data/signals, the buckle 585 (really a housing that includes the clasps, contacts, and wires, and might also include oscillator, demodulation electronics, etc.) further includes a wireless communications component (not shown).

In embodiments, three-ways buckles may be used in place of the two-way buckles 550 and 585 depicted in the Figures. According to this embodiment, for each of the buckles, the two side clasps fasten the ECG band 520 or respiratory band 584 to the respective housing while a third band (not shown) with a conductive component is connected on each end thereof to a respective third clasp of each housing.

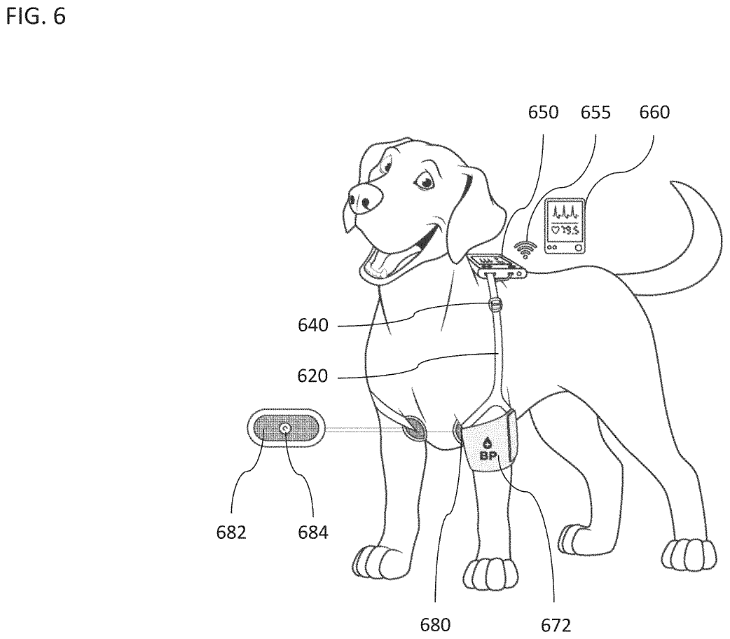

Yet another configuration is shown in FIG. 6. In example embodiments, the band material 620 (also referred to as a ‘strip’ or ‘band’ or ‘strap’) is an elastic material that easily stretches. The band is wrapped around the thorax and/or abdomen and/or limbs and clasped in place with a connecting piece (buckle) 640. The buckle and band (also referred to herein as a harness) are attached mechanically to a data logger/monitor module 650. In embodiments, the strap runs through one or more eyelets of the data logger or is threaded through a channel running the width of the device. In some embodiments, the band includes a conductive element. In such embodiments, the connection to the data logger may be electrical as well as mechanical. The data logger and buckle may be collocated in a single housing that mechanically (and in some cases electrically) couples the band to the module.

The data logger 650 is held in place by the strap 620 in a position on the animal that is both secure and not easily dislodged by the animal. The logger includes a wireless communications module for transmitting wireless signals 655 indicative of the vital signs data gathered by the data logger. The data is transmitted wirelessly to a remote device 660 for monitoring. The remote device may be collocated with the animal and/or remotely located. The signal may be transmitted via Wi-Fi and/or any other relevant wireless protocol.

The data logger 650 is in communication with at least one vital signs sensor disposed on the animal. FIG. 6 depicts a plurality of sensors. These sensors are merely exemplary both in their location and in their type. More or less or different sensors may be used.

That being said, common vital signs sensors used for monitoring veterinary patients include, but are not limited to: heart rate monitor, blood pressure monitor (cuff) 672, SpO2 (Oxygen saturation) monitor, temperature sensor (e.g., core body temperature, skin temperature and/or ambient temperature), one or more ECG sensors 680. ECG and temperature sensors are preferably located on or in proximity to the underarm or underarms of one or both forelegs. An ECG patch 680 generally needs to be adhered to the skin of the animal with a conductive (and preferably temporarily adhesive) material such as a gel. The ECG patch is also referred to herein as a sensor, and/or electrode as the ECG sensor is usually provided in the form of a bio adhesive patch 682 with a sensor/electrode 684 disposed in the center thereof. The patch is often coated with an adhesive layer as well as conductive material. In some embodiments, the adhesive layer is also the conductive material. In some embodiments, a patch may include other or additional sensors, e.g., such as those listed above.

Additional sensors that are not vital signs sensors may also be connected to the module. Examples of physical or physiological parameters which are not “vital signs” include but are not limited to: body motion/acceleration, spatial orientation, and other motion sensors, as well as ambient temperature or relative humidity (RH) sensors.

The sensors are in electrical communication (by wired or wireless means) with the logger. In example embodiments, a wire or lead from the BP cuff, temperature, SPO2, heartrate, and/or ECG sensor can be run along (on top of, under, inside, proximal to) the strap 620 to the data logger. In embodiments, the sensor or sensors are in wireless communication with the data logger. In embodiments, some sensors are in wire communication and other are in wireless communication.

The terms “processor” and/or “processing unit” as used herein may additionally or alternatively refer to a controller. Such processor may relate to various types of processors and/or processor architectures including, for example, embedded processors, communication processors, graphics processing unit (GPU)-accelerated computing, soft-core processors and/or embedded processors.

According to some embodiments, the device includes one or more types of computer-readable storage media. One type of memory is referred to herein as a working memory, such as memory 154. This type of memory may for example be in the form of a static random access memory (SRAM), dynamic random access memory (DRAM), read-only memory (ROM), cache or flash memory. Working memory may, for example, process temporally-based instructions.

Another type of memory is long-term memory, also referred to herein as storage, such as storage 156. Long-term memory may, for example, include a volatile or non-volatile computer storage medium, a hard disk drive, a solid state drive, a magnetic storage medium, a flash memory and/or other storage facility. A hardware memory facility may for example store a fixed information set (e.g., software code) including, but not limited to, a file, program, application, source code, object code, and the like.

A communication module, e.g., input/output (I/O) module 160, may for example include I/O device drivers and necessary software, firmware, and/or hardware for receiving and interpreting electrical impulses and/or signals from sensors, the ECG band, and/or the respiration band. A device driver may for example, interface with a keypad or to a USB port.

A remote communications module, such as wireless communications module 158, may include, for example, network interface drivers (not shown) for enabling the transmission and/or reception of signals carrying data over a network (not shown).

A network interface driver may for example execute protocols for the Internet, or an Intranet, Wide Area Network (WAN), Local Area Network (LAN) employing, e.g., Wireless Local Area Network (WLAN)), Metropolitan Area Network (MAN), Personal Area Network (PAN), extranet, 2G, 3G, 3.5G, 4G including for example Mobile WIMAX or Long Term Evolution (LTE) advanced, and/or any other current or future communication network, standard, and/or system.

While the invention has been described with respect to a limited number of embodiments, it will be appreciated that many variations, modifications and other applications of the invention may be made. Therefore, the claimed invention as recited in the claims that follow is not limited to the embodiments described herein.

What is claimed is:

1. A veterinary vital signs monitoring system, comprising:
(A) two stretchable bands, each having an electrically conductive component;
(B) a housing including two fasteners with electrically conductive contacts, each fastener adapted to receive, and lock therein a portion of one of the bands, the conductive contacts adapted to make an electrical connection with electrically conductive components of the bands; and
(C) a module housed in the housing and in electrical communication with the conductive contacts, the module adapted to receive signals relating to vital signs of an animal on which the bands are mounted, via the conductive components thereof.

2. The system of claim 1, wherein the signals received via the conductive component are electrical signals produced by a heart of the animal.

3. The system of claim 2, wherein a conductive gel or paste is adapted to be applied between a portion of each band and any two separate locations on the animal to enhance conductivity of the electrical signals between the heart and the conductive component of the band.

4. The system of claim 2, wherein the electrical signals are adapted to be processed into an electrocardiogram (ECG) by the module.

5. The system of claim 1, further comprising at least one vital signs sensor in wired or wireless communication with the module or embedded therein, the at least one sensor selected from the group including: an oxygen saturation (SPO2) sensor, a blood pressure sensor, and a temperature sensor.

6. The system of claim 1, further comprising at least one non-vital sensor in wired or wireless communication with the module or embedded therein.

7. The system of claim 1, wherein the module is adapted to fit between shoulder blades of the front legs of a four-legged animal.

8. The system of claim 1, wherein the housing is adapted to be held in place by the stretchable bands placed around or near the chest area or forelegs of the animal.

9. The system of claim 1, wherein the housing is adapted to be secured to the animal.

10. The system of claim 1, further comprising:
a Respiratory Inductance Plethysmography (RIP) sensor comprising:
respiration sensing belt comprised of a non-conductive stretchable material and conductive material disposed in or on the stretchable material, and
a coupling piece adapted to mechanically and electrically couple together two ends of the respiration sensing belt once positioned around a torso of the animal; and
an electronic interface adapted to obtain digital waveforms from the respiration sensing belt, via the coupling piece;
wherein the RIP sensor is in wired or wireless communication with the module or coupled to the housing.

11. The system of claim 10, wherein the coupling piece includes two electrical contacts and wherein fastening ends of the respiration band to the coupling piece brings the conductive material into secure and unmoving contact with the electrical contacts.

12. The system of claim 1, wherein each of the stretchable bands is pre-cut or cut from a larger roll of a same material.

13. The system of claim 1, wherein the housing further includes at least one additional conductive component.

14. The system of claim 1, wherein the module includes a wireless communication module for wirelessly transmitting the received signals to a remote computer.

15. A method of monitoring an animal, the method comprising:
providing two stretchable bands, each band adapted to be coupled to the animal on one end thereof;
providing a housing with fasteners for the stretchable bands;
installing a module in housing;
positioning the module between shoulder blades of forelegs of the animal, securing the bands to the animal so that at least a portion of each band is in contact with a separate respective area of skin of the animal;
fastening loose ends of the bands to the fasteners of the housing;
providing at least one sensor on the animal, the at least one sensor being in electrical communication with the module;
receiving sensor data at the module from the at least one sensor.

16. The method of claim 15, further comprising:
wirelessly transmitting the received sensor data to a remote monitoring station.

17. The method of claim 15, wherein each material band has an electrically conductive material;
wherein the fasteners include electrical contacts in electrical communication with the module; and
wherein fastening the two material bands to the fasteners mechanically couples the bands to the housing and electrically couples the conductive material to the module.

18. The method of claim 15, wherein at least one sensor is selected from the group including: a temperature sensor, an oxygen saturation sensor, a blood pressure sensor, and a Respiratory Inductance Plethysmography (RIP) sensor.

19. The method of claim 18, wherein the RIP sensor includes:
a respiration sensing belt comprised of a non-conductive stretchable material and conductive material disposed in or on the stretchable material, and
a coupling piece adapted to mechanically and electrically couple together two ends of the respiration sensing belt once positioned around a torso of the animal;
wherein the RIP sensor is in wired or wireless communication with the module.

20. The method of claim 17, further comprising:
applying a conductive gel or paste to the separate respective areas of skin of the animal prior to securing the bands to the animal such that the conductive components are in electrical contact with the areas of skin to enable electrical signals produced by the heart of the animal to be conducted to the module via the conductive components.

21. The method of claim 17, wherein at least one additional sensor is attached to the animal and in electrical communication with the module.

22. The method of claim 21, wherein at least one additional sensor is selected from the group including: a temperature sensor, an oxygen saturation sensor, a blood pressure sensor, and a Respiratory Inductance Plethysmography (RIP) sensor.

23. The method of claim 22, wherein the RIP sensor includes:
a respiration sensing belt comprised of a non-conductive stretchable material and conductive material disposed in or on the stretchable material, and
a coupling piece adapted to mechanically and electrically couple together two ends of the respiration sensing belt once positioned around a torso of the animal;
wherein the RIP sensor is in wired or wireless communication with the monitoring module.
